(19) United States
(12) Patent Application Publication
(10) Pub. No.: US 2021/0098122 A1
(43) Pub. Date: Apr. 1, 2021

(54) CONVERSION OF A MAGNETIC RESONANCE (MR) MEASURING PROTOCOL

(71) Applicant: Siemens Healthcare GmbH, Erlangen (DE)

(72) Inventor: Stefan Meyer, Langensendelbach (DE)

(73) Assignee: Siemens Healthcare GmbH, Erlangen (DE)

(21) Appl. No.: 17/060,430

(22) Filed: Oct. 1, 2020

(30) Foreign Application Priority Data
Oct. 1, 2019 (EP) 19200780.5

(51) Int. Cl. G16H 40/40; G01R 33/54; G16H 30/20; G06F 8/71

(52) U.S. Cl. CPC G16H 40/40; G06F 8/71; G16H 30/20; G01R 33/543

(57) ABSTRACT

Techniques are disclosed for the conversion of an MR measuring protocol, which predetermines parameters with associated parameter values. The parameter values configure a measurement by means of a software version of a first magnetic resonance system. The technique includes a syntactic conversion of the MR measuring protocol, in which the structure of the measuring protocol is adjusted to an older software version of a second magnetic resonance system. Furthermore, the technique includes a semantic conversion of the syntactically converted MR measuring protocol, by dependencies on parameter values of specific parameters of the syntactically converted MR measuring protocol being taken into account with respect to the older software version.

### CROSS-REFERENCE TO RELATED APPLICATIONS

The present application claims the benefit of the filing date of European patent application no EP 19200780.5, filed on Oct. 1, 2019, the contents of which are incorporated herein by reference in their entirety.

### TECHNICAL FIELD

The present disclosure relates to the conversion of an MR measuring protocol, which predetermines parameters with associated parameter values, which configure a measurement by means of a magnetic resonance system.

### BACKGROUND

With magnetic resonance systems or MR (magnetic resonance) tomography systems, the totality of all parameters, which influence or configure the imaging measurement by means of the magnetic resonance system, is stored in MR measuring protocols or parameter sets. Such MR measuring protocols are typically supplied together with the rollout of a specific software version, which is optimally attuned to the corresponding magnetic resonance system.

Furthermore, clinical users of the magnetic resonance system may also create and store MR measuring protocols (known as user protocols) for their measurements. If a magnetic resonance system is later updated to a new software version, these user protocols are converted to ensure that they also have parameter settings that are consistent with the current software version.

With users of magnetic resonance systems that operate with numerous magnetic resonance systems, different software versions are occasionally used for the same or different magnetic resonance systems. For these users, there is a desire to be able to use an MR measuring protocol, which has been generated for any software version, for other software versions that run on any magnetic resonance systems.

### SUMMARY

The present disclosure therefore has the object of providing for an MR measuring protocol, which is generated for a new software version, which can also be used for an older software version of a magnetic resonance system.

According to the disclosure, this object is achieved by a method for converting an MR measuring protocol, a device for converting an MR measuring protocol, a magnetic resonance system, a computer program product, and by an electronically readable data storage medium, each being described throughout the disclosure and in the claims, in various aspects.

Within the scope of the present disclosure, provision is made for a method for converting an MR measuring protocol. Here, an MR measuring protocol predetermines parameters (i.e. includes predetermined parameters) with associated parameter values, by means of which a measurement is configured by means of a new software version by a first magnetic resonance system. In an aspect, the method comprises the following steps:

Syntactic conversion of the measuring protocol. Here, the structure of the MR measuring protocol is automatically adjusted to an older software version of a second magnetic resonance system.

Semantic conversion of the syntactically converted MR measuring protocol. With this semantic conversion, dependencies of the parameter values of specific parameters with respect to the older software version are automatically taken into account. In this step, checks are carried out in particular to determine whether the parameter values of the syntactically-converted MR measuring protocol are still semantically correct within the corresponding application. If this is not the case, the parameter values are automatically changed accordingly.

The semantic conversion adjusts the content of the MR measuring protocol (i.e. the parameter values) to the properties implemented in the old software version, by the dependencies between the parameters including their parameter values being taken into account.

The aforementioned object is achieved by an MR measuring protocol written for a new software version also being adjusted by the conversion to an older software version. Aside from the known conversion, in which MR measuring protocols are converted from older software versions to newer software versions, it is now also possible in accordance with the disclosure for an MR measuring protocol of a newer software version to be converted to an older software version. As a result, it is advantageously possible to import an MR measuring protocol of a newer software version onto a magnetic resonance system with an older software version and to use the same. Users can therefore also use an MR measuring protocol, which is written for a specific software version, for other software versions, irrespective of whether this software version is a newer or an older software version.

A new or newer software version is understood herein to mean a software version which has been created after an older software version in terms of time, and has software features, for instance, which the older software version does not have. The new software version is adjusted in particular to the current state of the art, at the point in time of its creation, which is newer than the state of the art that was applicable when the older software version was created.

Conversion rules are predetermined with respect to the conversion of the MR measuring protocol. Here, each of these conversion rules adjusts a parameter of the MR measuring protocol to the older software version. In this way, this adjustment of a parameter can comprise a renaming and deletion of this parameter and/or a change in the parameter value. These conversion rules are, for instance, used in the step of syntactical conversion, wherein by way of example the conversion rules may (e.g. only) realize a search and replace functionality with respect to the parameters.

These conversion rules can be stored, for instance, together with MR measuring protocols for the new software version. In other words, the conversion rules for the conversion are exported from the new to the old software version together with the MR measuring protocol and stored with the (new) MR measuring protocol.

By the conversion rules, together with the MR measuring protocols being stored for the new software version, it is ensured that the conversion rules exist if the MR measuring protocols for the new software version are converted to the older software version.

In accordance with the disclosure, it is also possible to carry out the syntactical conversion without conversion rules. This results for instance in, according to the old software version, unknown parameters in the MR measuring protocol not being able to be read in and parameters, which according to the old software version, are expected in the MR measuring protocol but are not available, being reinitialized. Both previously outlined cases result for instance in these parameters being initialized in syntactically-converted MR measuring protocols with default values. For instance, inconsistencies occurring as a result can then be triggered in the following semantic conversion.

The step of semantic conversion is performed identically irrespective of whether the syntactic conversion operates with or without conversion rules.

Both possibilities (syntactic conversion with or without conversion rules) result in a successful conversion of the MR measuring protocol to the older software version.

The first magnetic resonance system can be the second magnetic resonance system, which means that the first and the second magnetic resonance system may be the same magnetic resonance system. It is also possible for the first and the second magnetic resonance system to be of the same type of magnetic resonance system. In these cases, the new and the old software version apply to the same hardware or the same type of a magnetic resonance system.

It is also possible in accordance with the aspects described herein, however, for the first magnetic resonance system to differ from the second magnetic resonance system in terms of its hardware, so that the first magnetic resonance system is newer than the second magnetic resonance system with respect to a manufacturing date. In this case, the new software version may be adjusted to the hardware of the first magnetic resonance system, while the old software version may be adjusted to the hardware of the second magnetic resonance system that differs from the hardware of the first magnetic resonance system.

In an aspect, the syntactic conversion comprises the following steps:

Removing parameters of the MR measuring protocol which are not supported by the old software version.

Inserting new parameters of the MR measuring protocol, which are supported by the old software version but not by the new software version.

Setting the new parameters to a standard (e.g. predetermined) value.

Taking over (e.g. using or establishing) parameter values of parameters of the MR measuring protocol, which, with the old software version, have a different name to that in the new software version.

In an aspect, the semantic conversion comprises the following steps:

Generating a standard (e.g. predetermined) protocol of the old software version as a target protocol.

Copying parameters of the MR measuring protocol to the associated parameters of the target protocol. For instance, in this step, parameter values of the parameters of the MR measuring protocol are assigned to the associated parameters of the target protocol. Here, limits and dependencies of the parameters of the target protocol are taken into account with respect to the old software version.

At the end of the semantic conversion, the target protocol should correspond to the MR measuring protocol, which is converted with respect to the old software version.

With the previously outlined steps of semantic conversion, all parameters of the target protocol are checked repeatedly to determine whether the parameter values of these parameters comply with the limits and dependencies with respect to the old software version. If this is not the case (i.e. at least one of the parameter values of the parameters of the target protocol breaches (e.g. does not comply with) one of the limits or one of the dependencies with respect to the old software version), the parameter values of the parameters of the target protocol are set so that the parameter values or parameters no longer breach the limits and dependencies with respect to the old software version. This is repeated, for example, until all parameter values comply with the limits and dependencies with respect to the old software version.

Within the scope of the present disclosure, provision is also made for a device for converting an MR measuring protocol. Herein, the device according to the disclosure comprises a computer unit (e.g. one or more processors, a controller, control circuitry, etc.) and a storage means. The device is configured to store the MR measuring protocol completely or partially in the storage means, to convert the MR measuring protocol syntactically with the aid of the computer unit, and to semantically convert the syntactically-converted MR measuring protocol by means of the computer unit, by dependencies of the parameter values of specific parameters being taken into account with respect to the old software version.

The advantages of the device aspects described herein essentially correspond to the advantages of the method aspects described herein, which are explained above in detail so that it is possible to dispense with repetition here.

Furthermore, in an aspect a magnetic resonance system is provided, which comprises the previously described device.

Furthermore, the aspects described herein describe a computer program product, in particular software, which can be loaded into a memory (e.g. a non-transitory computer-readable medium) of a programmable control device or a computer unit of a magnetic resonance system. This computer program product can be used to implement all, or a variety of, the embodiments described above of the method aspects when the computer program product is running in the control device. In this context, the computer program product may require program means, e.g. libraries and auxiliary functions, for implementing the relevant aspects of the method(s) described herein. In other words, the computer program product aspects are intended to provide protection for software that can be used to implement one of the above-described aspects of the method(s) described throughout the disclosure, and/or implements said aspects. Said software may be a source code (e.g. C++), which still needs to be compiled and linked or just needs to be interpreted, or an executable software code, which for execution only needs to be loaded into the relevant computer unit or control device.

Finally, the present disclosure discloses an electronically readable data storage medium (e.g. a non-transitory computer-readable medium), for instance a DVD, a magnetic tape, a hard disk, or a USB stick, on which is stored electronically readable control data, in particular software (see above). When this control data (software) is read from the data storage medium and stored in a control device and/or computer unit of a magnetic resonance system, all the aspects according to the disclosure of the above-described method can be performed.

Advantageously, MR measuring protocols, which are designed for new software versions, can also be imported onto and used in magnetic resonance systems which operate with an older software version. As a result, a user of this magnetic resonance system with older software is spared the task of having to reapply corresponding MR measuring protocols with respect to the old software.

The present disclosure thus advantageously enables a backward compatibility of MR measuring protocols.

The present disclosure enables users who use numerous magnetic resonance systems with different software versions to export an MR measuring protocol from any magnetic resonance system and import it onto all other magnetic resonance systems.

### BRIEF DESCRIPTION OF THE DRAWINGS/FIGURES

The present disclosure is described in detail below using embodiments according to the disclosure with reference to the figures:

FIG. 1 shows an example flow for a conversion process, in accordance with one or more aspects of the present disclosure;

FIG. 2 shows an example magnetic resonance system, in accordance with one or more aspects of the present disclosure.

### DETAILED DESCRIPTION

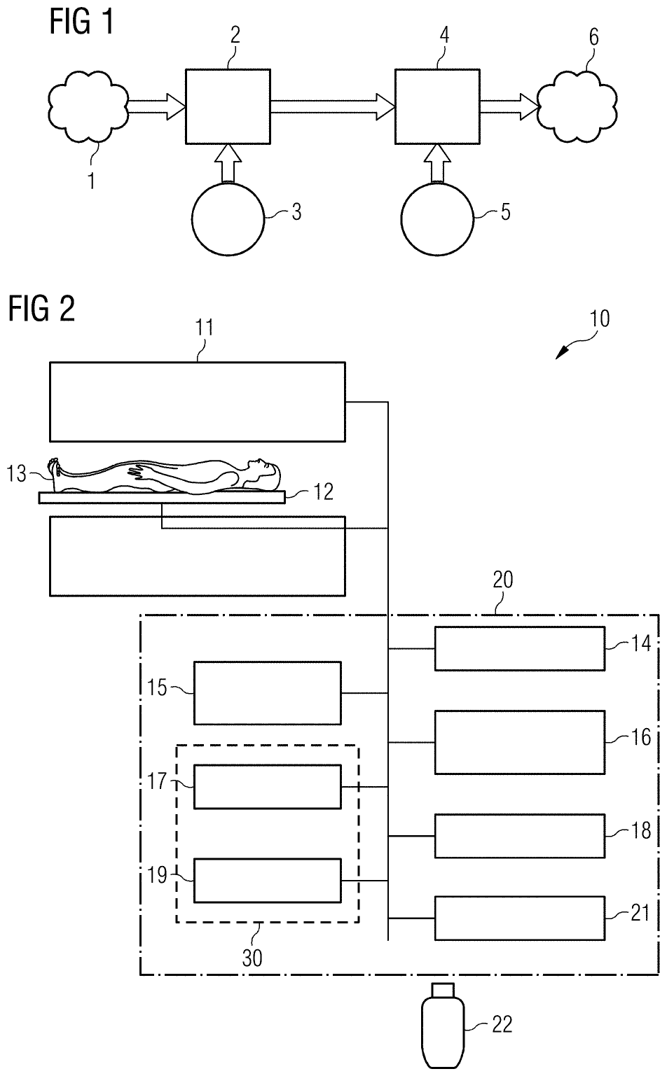

FIG. 1 shows example flow for a conversion process, in which an MR measuring protocol 1 of a new software version is converted into an MR measuring protocol 6 of an old software version.

In a first step, a syntactic conversion 2 of the MR measuring protocol of the new software version takes place by using conversion rules 3. This first step can optionally also be carried out without conversion rules 3.

In a second step, the syntactically-converted MR measuring protocol is then subjected to a semantic conversion 4, wherein parameter dependencies 5 are taken into account. The result of this second step is the converted MR measuring protocol 6 for the old software version.

The conversion may run automatically and is outlined in FIG. 1, and is explained in further detail below.

The data structure of the MR measuring protocol to be converted is firstly adjusted to the data structure of the old software version. During this adjustment, parameters which are no longer available in the old software version are removed, in the old software version, but parameters which are not available in the new software version are inserted and set to a default value, and parameter values for unnamed parameters (i.e. parameters which bear a different name in the old software version than in the new software version, but have the same functionality) are taken over (e.g. used or established). If sequences in the old software version are replaced, then a reference to the sequence, which is used by the converted protocol, is likewise updated.

A default MR measuring protocol of the sequence, which is used by the converted MR measuring protocol, is then generated as a new (target) MR measuring protocol, and attempts are made to apply the coil context of the MR protocol to be converted to the target MR measuring protocol. If coils that are selected in the MR measuring protocol to be converted are not available in the old software version (e.g. an old magnetic resonance system), a compatible coil with respect to the old software version (e.g. the old magnetic resonance system) is sought and attempts are made to adjust the corresponding coil elements. If no compatible coil is found, a body coil is used. This can result in adjustments to dependent parameters (e.g. parallel acquisition techniques (PAT) factor, normalization filter of a prescan, etc.).

Finally, each parameter value in a number of loops is copied from the MR measuring protocol to be converted into the target measuring protocol, wherein limits and dependencies of these parameters are observed with respect to the old software version. With each of these cycles, other parameters can be adjusted in order, as a result, to comply with the parameter dependencies of the old software version.

At the end of the conversion, the target MR measuring protocol corresponds to the converted MR measuring protocol for the old software version.

The conversion of an MR measuring protocol from a new software version to an old software version is explained in detail below on the basis of an illustrative example.

TABLE 1
MR measuring protocol according to new software version

converter=%MEASCONST%/ConverterList/Prot_Converter.txt ulVersion = 61010001
tSequenceFileName = “%SiemensSeq%\space”
tProtocolName = “t2_spc_rst_cor_p3_trig_384_iso” tdefaultEVAProt =
“%SiemensEvaDefProt%\Inline\Inline.evp” lMeasPause = 30000000
alTR[0] = 2400000
sKSpace.lBaseResolution = 384
sKSpace.ucMultiSliceMode = 1
sPat.ucPATMode = 2

TABLE 2
Conversion rules

[61010002 −> 61010001] // VersionNew −> VersionOld
//Replaces the inner-lying sequence (==Application)
if (tSequenceFileName == “%SiemensSeq%\space”) set (tSequenceFileName,
“%SiemensSeq%\space2”)
// Removes the parameter
delete (lMeasPause)
//Rename a parameter
rename(alTR, alRepetitionTime)
//Change a parameter value as a function of other parameters (e.g. change due to a feature,
which exists in the old, but not in the new software version)
if (sKSpace.ucMultiSliceMode == 1) set (sKSpace.lBaseResolution, 512)
//Set a parameter value (e.g. activate a feature which exists in the old but not in the new
software version)
if (sPat.ucPATMode == 2) set (sPat.ucPATMode, 4)
//Add a parameter
default(newParameter, 42)

TABLE 3
MR measuring protocol converted into old software version.

tSequenceFileName = “%SiemensSeq%\space2”
tProtocolName = “t2_spc_rst_cor_p3_trig_384_iso” tdefaultEVAProt =
“%SiemensEvaDefProt%\Inline\Inline.evp” alRepetitionTime[0] = 2400000
sKSpace.lBaseResolution = 512
sKSpace.ucMultiSliceMode = 1
sPat.ucPATMode = 4
newParameter = 42

Table 1 shows an MR measuring protocol, which is written into a new software version. This MR measuring protocol should be converted to an old software version with the aid of the conversion rules shown in Table 2. The result of this conversion is shown in Table 3 in the form of the converted MR measuring protocol.

FIG. 2 shows an example magnetic resonance system 10, which has a magnet 11 for generating a polarization field B0, wherein an examination person 13 arranged on a couch 12 is moved into the magnet 11 to receive magnetic resonance signals spatially encoded there from the examination person 13. The coils used for the signal acquisition, such as a whole body coil or local coils, are not shown for reasons of clarity.

The magnetic resonance system 10 also has a controller 20 (e.g. one or more processors and/or a control computer), which can be used to control the magnetic resonance system 10. The controller 20 has a gradient control unit 15 for controlling and switching the necessary magnetic field gradient. An RF control unit 14 is provided for controlling and generating the RF pulses for deflecting the magnetization. An image sequence controller 16 controls the sequence of magnetic field gradients and RF pulses and thus indirectly the gradient control unit 15 and the RF control unit 14. By means of an input unit 18, an operator can control the magnetic resonance system and MR images and other information necessary for the controller 20 can be displayed on a display unit 21.

A computer unit 17 with at least one processor unit (not shown, but may include one or more processors and/or processing circuitry) and storage means 19 (e.g. a non-transitory computer-readable medium) are provided for controlling the different units in the controller 20. In addition, program modules or programs, which can control the running of the magnetic resonance system 10 when they are executed by the controller 20, the computer unit 17, and/or or its processor unit, can be stored in the storage means 19, for instance. The computer unit 17 and the storage means 19 are elements of a device 30, which is therefore an integral part of the aspects described herein that may be implemented via the magnetic resonance system 10.

Furthermore, a data storage medium or USB stick 22 is shown, on which a software packet or computer program product may be stored.

The various functional blocks, apparatuses, modules, units, components of physical or functional units, etc., as shown in the drawings and described herein may be implemented unless otherwise noted via any suitable number and type of computer processors, hardware components, the execution of software algorithms, or combinations thereof, and thus may alternatively be referred to as a “unit,” “system,” “circuitry,” or “device.”

What is claimed is:

1. A method for converting a magnetic resonance (MR) measuring protocol that includes predetermined parameters with associated parameter values that configure MR data measurement with a first magnetic resonance system in accordance with a first software version, the method comprising:
performing, via one or more processors, a syntactic conversion of the MR measuring protocol to provide a syntactically-converted MR measuring protocol by adjusting a structure of the MR measuring protocol to provide a second software version that is associated with a second magnetic resonance system; and
performing, via one or more processors, a semantic conversion of the syntactically-converted MR measuring protocol using dependencies on parameter values associated with parameters of the syntactically-converted MR measuring protocol with respect to the second software version.

2. The method as claimed in claim 1, wherein performing the syntactic conversion of the MR measuring protocol includes using predetermined conversion rules, each of the conversion rules adjusting a parameter associated with the MR measuring protocol to the second software version.

3. The method as claimed in claim 2, wherein the conversion rules are stored in a storage device with MR measuring protocols associated with the first software version.

4. The method as claimed in claim 1, wherein the first magnetic resonance system is identical to the second magnetic resonance system.

5. The method as claimed in claim 1, wherein the first magnetic resonance system differs from the second magnetic resonance system with respect to hardware.

6. The method as claimed in claim 1, wherein performing the syntactic conversion of the MR measuring protocol comprises:
removing parameters of the MR measuring protocol that are not supported by the second software version;
inserting new parameters associated with the MR measuring protocol that are supported by the second software version but not by the first software version;
setting the new parameters to a predetermined value; and
using parameter values from parameters of the MR measuring protocol that have a different name in the second software version than in the first software version.

6. The method as claimed in claim 1, wherein performing the semantic conversion of the MR measuring protocol comprises generating a predetermined protocol of the second software version as a target protocol by copying parameters of the MR measuring protocol into the associated parameters of the target protocol,
wherein limits and dependencies of the parameters of the target protocol are used with respect to the second software version such that the target protocol corresponds to the MR measuring protocol that is converted with respect to the second software version.

7. The method as claimed in claim 6, further comprising:
checking, via one or more processors, the parameters of the target protocol to determine whether predetermined limits and dependencies are complied with respect to the second software version; and
when one of the parameters of the target protocol does not comply with the predetermined limits and dependencies, adjusting the parameters of the target protocol such that the respective parameter complies with the predetermined limits and dependencies.

8. A device for converting magnetic resonance (MR) measuring protocol that includes predetermined parameters with associated parameter values that configure MR data measurement with a first MR system in accordance with a first software version, comprising:
a memory configured to store at least a portion of the MR measuring protocol; and
one or more processors configured to:
syntactically convert the MR measuring protocol to provide a syntactically-converted MR measuring protocol by adjusting a structure of the MR measuring protocol to provide a second software version that is associated with a second magnetic resonance system; and
semantically convert the syntactically-converted MR measuring protocol using dependencies on parameter values associated with parameters of the syntactically-converted MR measuring protocol with respect to the second software version.

9. The device as claimed in claim 8, wherein the device is part of a magnetic resonance system.

10. A non-transitory computer-readable medium having instructions stored thereon that, when executed by one or more processors associated with a magnetic resonance (MR) system, cause the MR system to convert a MR measuring protocol that includes predetermined parameters with associated parameter values that configure MR data measurement with a first MR system in accordance with a first software version by:
syntactically converting the MR measuring protocol to provide a syntactically-converted MR measuring protocol by adjusting a structure of the MR measuring protocol to provide a second software version that is associated with a second magnetic resonance system; and
semantically converting the syntactically-converted MR measuring protocol using dependencies on parameter values associated with parameters of the syntactically-converted MR measuring protocol with respect to the second software version.
